(19) United States
(12) Patent Application Publication
(10) Pub. No.: US 2014/0170231 A1
(43) Pub. Date: Jun. 19, 2014

(54) PAIN RELIEF COMPOSITIONS AND METHODS

(71) Applicant: Derjers International, Detroit, MI (US)

(72) Inventors: Axel Cooley, Detroit, MI (US); Derrick Cooley, Detroit, MI (US); John Cooley, Vance, MS (US)

(73) Assignee: Derjers International, Detroit, MI (US)

(21) Appl. No.: 14/108,562

(22) Filed: Dec. 17, 2013

Related U.S. Application Data
(60) Provisional application No. 61/738,224, filed on Dec. 17, 2012.

(51) Int. Cl. A61K 36/54; A61K 36/185; A61K 31/015; A61K 35/56; A61K 36/31; A61K 31/235; A61K 31/555

(52) U.S. Cl. CPC A61K 36/54; A61K 31/235; A61K 36/185; A61K 31/555; A61K 35/57; A61K 36/31; A61K 31/015
USPC 424/522

(57) ABSTRACT

An analgesic composition useful for soothing body aches, pains and sores. The analgesic composition can include methyl salicylate, turpentine oil, camphor oil, sassafras oil, beta carotene, emu oil, oleoresin capsicum, chlorophyllin, and mustard oil. Methods for soothing body aches. The methods include administering an analgesic composition topically at a location of a body ache.

### CROSS REFERENCE TO RELATED APPLICATIONS

This application claims the benefit of U.S. provisional application 61/738,224 filed Dec. 17, 2012, the full disclosure of which is hereby incorporated by reference.

### FIELD

The present disclosure relates to pain relief compositions and methods for soothing body aches.

### BACKGROUND

This section provides background information related to the present disclosure which is not necessarily prior art.

It is commonplace for analgesic ointments to be purchased over the counter. Such ointments are often used by athletes who generate sore muscles through physical activity. Patients suffering from various ailments, such as arthritis, also typically use analgesic ointments. Despite their widespread use, several improvements in their composition may be made.

### SUMMARY

This section provides a general summary of the disclosure, and is not a comprehensive disclosure of its full scope or all of its features.

The present technology provides analgesic compositions. The compositions comprise active ingredients and inactive ingredients. The active ingredients include methyl salicylate, turpentine oil, camphor oil, menthol, sassafras oil, beta carotene, emu oil, oleoresin capsicum, chlorophyllin, and mustard oil. Some compositions include all of the active ingredients, and other compositions do not include all of the active ingredients. The active ingredients can be present in various concentrations. For example, various compositions comprise from about 2% to about 6% methyl salicylate, from about 2% to about 6% turpentine oil, from about 2% to about 6% camphor oil, from about 2% to about 6% menthol, from about 2% to about 6% sassafras oil, from about 2% to about 6% beta carotene, from about 0.05% to about 2% emu oil, from about 0.1% to about 2% oleoresin capsicum, from about 0.05% to about 1% chlorophyllin, and from about 0.01% to about 1% mustard oil, wherein all percentages are by weight, based on the total weight of the composition.

The inactive ingredients include various pharmaceutical carriers. In some embodiments, the composition is a cream. Where the composition is a cream, the pharmaceutical carriers can comprise white petrolatum, and medium wax. The pharmaceutical carriers can be present in various concentrations. For example, various compositions comprise from about 60% to about 82% white petrolatum, and from about 2% to about 6% medium wax, wherein all percentages are by weight, based on the total weight of the composition.

In various embodiments, the composition is a spray. In a sprayable composition, the pharmaceutical carriers can comprise a polysorbate, glycerin, a poloxamer, alcohol, and water. The pharmaceutical carriers can be present in various concentrations. For example, 0.1-5% polysorbate, 2%-20% glycerin, 2%-20% poloxamer, 3%-15% alcohol, and 10%-40% water.

The present technology also provides methods for soothing body aches. The method comprises administering an analgesic composition topically at a location of a body ache to a subject in need thereof. The subject can administer the analgesic composition when experiencing at least one body ache or when the development of a body ache is anticipated. The analgesic composition can be administered by rubbing or by spraying. The analgesic composition can comprise active ingredients and inactive ingredients. The active ingredients can be methyl salicylate, turpentine oil, camphor oil, menthol, sassafras oil, beta carotene, emu oil, oleoresin capsicum, chlorophyllin, and mustard oil.

Where the analgesic composition is a cream, which is administered by rubbing, the inactive ingredients can be white petrolatum, medium wax, and beta carotene. Where the analgesic composition is a solution, which is administered by spraying, the inactive ingredients can be a polysorbate, glycerin, a poloxamer, an alcohol, and water.

Further areas of applicability will become apparent from the description provided herein. The description and specific examples in this summary are intended for purposes of illustration only and are not intended to limit the scope of the present disclosure.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” and “including,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

The present technology provides analgesic compositions. The compositions comprise active ingredients and inactive ingredients. Various active ingredients produce a heating sensation, and others produce a cooling sensation. Collectively, the active ingredients have been found to produce an exceptionally soothing sensation when administered to a subject.

The active ingredients comprise methyl salicylate, turpentine oil, camphor oil, menthol, sassafras oil, beta carotene, emu oil, oleoresin capsicum, chlorophyllin, and mustard oil. Some compositions include all of the active ingredients, and other compositions do not include all of the active ingredients. For example, the active ingredients of the composition can comprise methyl salicylate, turpentine oil, camphor oil, sassafras oil, beta carotene, and oleoresin capsicum. In another embodiment, the active ingredients comprise methyl salicylate, turpentine oil, camphor oil, sassafras oil, beta carotene, oleoresin capsicum, and emu oil. The active ingredients can also comprise methyl salicylate, turpentine oil, camphor oil, menthol, beta carotene, emu oil and oleoresin capsicum. Furthermore, the active ingredients can consist of methyl salicylate, turpentine oil, camphor oil, menthol, beta carotene, emu oil, and oleoresin capsicum, or the active ingredients can consist of methyl salicylate, turpentine oil, camphor oil, menthol, beta carotene, emu oil, and oleoresin capsicum, and sassafras oil. In various embodiments, the active ingredients of the composition comprise methyl salicylate, turpentine oil, camphor oil, sassafras oil, beta carotene, emu oil, and oleoresin capsicum, wherein the addition of at least one of menthol, chlorophyllin, and mustard oil is optional. In one embodiment, the active ingredients consist of methyl salicylate, turpentine oil, camphor oil, sassafras oil, beta carotene, emu oil, and oleoresin capsicum.

The active ingredients can be present in various concentrations. For example, various compositions comprise from about 2% to about 6% methyl salicylate, from about 2% to about 6% turpentine oil, from about 2% to about 6% camphor oil, from about 2% to about 6% menthol, from about 2% to about 6% sassafras oil, from about 2% to about 6% beta carotene, from about 0.05% to about 2% emu oil, from about 0.1% to about 2% oleoresin capsicum, from about 0.05% to about 1% chlorophyllin, and from about 0.01% to about 1% mustard oil, wherein all percentages are by weight, based on the total weight of the composition. The beta carotene can be added from a solution of 30% beta carotene in oil (w/w). In an exemplary embodiment, the compositions comprise 4.75% methyl salicylate, 4.75% turpentine oil, 4.75% camphor oil, 4.75% sassafras oil, 4.75% beta carotene, 1% emu oil, 0.59% oleoresin capsicum, 0.3% chlorophyllin, and 0.03% mustard oil. In various embodiments, the camphor oil is replaced with 4.75% menthol. In yet other embodiments, both camphor oil and menthol can be present at a concentration of 2.375%. These active ingredients can be obtained through common vendors known to those of ordinary skill in the art. In these and other embodiments described herein, and unless the context requires a different result, percentages represent weight percent, based on the total weight of the composition.

The inactive ingredients include various pharmaceutical carriers. In some embodiments, the composition is a cream. Where the composition is a cream, the pharmaceutical carriers can comprise white petrolatum, and medium wax. The pharmaceutical carriers can be present in various concentrations. For example, various compositions comprise from about 60% to about 85% white petrolatum, and from about 2% to about 6% medium wax, wherein all percentages are by weight, based on the total weight of the composition. Preferably, the compositions comprise 4.79% medium wax, and a sufficient amount of white petrolatum to bring the total w/w % to 100%. The medium wax can be a paraffin wax. These pharmaceutical carriers can be obtained through common vendors known to those of ordinary skill in the art.

In various embodiments, the composition is a sprayable solution. In a sprayable composition, the pharmaceutical carriers can comprise a polysorbate, glycerin, a poloxamer, an alcohol, and water. The pharmaceutical carriers can be present in various concentrations. For example, from about 0.1 to about 5% polysorbate, from about 2% to about 20% glycerin, from about 2% to about 20% poloxamer, from about 3% to about 15% alcohol, and from about 10% to about 40% water. Non-limiting examples of polysorbate are polysorbate 20 and polysorbate 80. An especially useful poloxamer is poloxamer 188. The alcohol can be any alcohol typically used in the art. Non-limiting examples of alcohol include ethanol, propanol, isopropanol, stearyl alcohol, and denatured alcohol, such as SD alcohol 40-B. These pharmaceutical carriers can be obtained through common vendors known to those of ordinary skill in the art.

The present technology also provides methods for soothing body aches. As used herein, the term “aches” includes sores and pains. The method comprises administering an analgesic composition topically at a location of a body ache to a subject in need thereof, wherein the subject experiences the body ache or anticipates a development of body ache. The analgesic composition is suitable for human subjects.

The analgesic composition can comprise active ingredients and inactive ingredients. The active ingredients comprise methyl salicylate, turpentine oil, camphor oil, sassafras oil, beta carotene, emu oil, oleoresin capsicum, chlorophyllin, and mustard oil. The active ingredients can be present in various concentrations. For example, various compositions comprise from about 2% to about 6% methyl salicylate, from about 2% to about 6% turpentine oil, from about 2% to about 6% camphor oil, from about 2% to about 6% menthol, from about 2% to about 6% sassafras oil, from about 2% to about 6% beta carotene, from about 0.05% to about 2% emu oil, from about 0.1% to about 2% oleoresin capsicum, from about 0.05% to about 1% chlorophyllin, and from about 0.01% to about 1% mustard oil, wherein all percentages are by weight, based on the total weight of the composition. In some embodiments, all of the ingredients are present, and in other embodiments, some of the ingredients are present. The technology provides for the substitution of camphor oil with menthol. Yet, in some embodiments, both camphor oil and menthol are present. The unique combination of active ingredients generates a soothing sensation when administered topically.

The analgesic composition can be administered as a cream or as a spray. Where the composition is a cream, the pharmaceutical carriers can comprise white petrolatum, medium wax. The pharmaceutical carriers can be present in various concentrations. For example, various compositions comprise from about 60% to about 85% white petrolatum, and from about 2% to about 6% medium wax, wherein all percentages are by weight, based on the total weight of the composition. The medium wax can be a paraffin wax.

Where the analgesic composition is a solution, which is administered by spraying, the inactive ingredients can be a polysorbate, glycerin, a poloxamer, an alcohol, and water. The pharmaceutical carriers for the solution can be present in various concentrations. For example, the concentrations can be from about 0.1 to about 5% polysorbate, from about 2% to about 20% glycerin, from about 2% to about 20% poloxamer, from about 3% to about 15% alcohol, and from about 10% to about 40% water. Non-limiting examples of polysorbate are polysorbate 20 and polysorbate 80. An especially useful poloxamer is poloxamer 188. The alcohol can be any alcohol typically used in the art. Non-limiting examples of alcohol include ethanol, propanol, isopropanol, stearyl alcohol, and denatured alcohol, such as SD alcohol 40-B.

The aches can be associated with arthritis, osteoarthritis, rheumatoid arthritis, tendonitis, carpal tunnel syndrome, fibromyalgia, a pulled muscle, a strained muscle, a back ache, a flu, diabetic neuropathy, cancer, or other ailment resulting in a body ache. The composition can relieve or prevent pain aches and stiffness, and can improve joint mobility. The formulations can be used when the subject experiences body aches and pains, or it can be used prophylactically when the subject anticipates a development of body aches or pains. Therefore, the composition can be administered prophylactically.

Body aches often result from strenuous physical activity, such as weight lifting, participation in sports, or laborious jobs. Accordingly, the analgesic composition can be administered before, during or after engaging in strenuous physical activity. It is also contemplated that the composition can be administered during a massage.

### EXAMPLES

The following specific examples are provided for illustrative purposes to demonstrate preferred embodiments.

I. Analgesic Compositions that can be Administered by Rubbing.

| | Composition | | | | | | | | |
|---|---|---|---|---|---|---|---|---|---|
| Component | 1 | 2 | 3 | 4 | 5 | 6 | 7 | 8 | 9 |
| methyl salicylate (% w/w) | 4.75 | 4.75 | 4.75 | 4.75 | 4.75 | 4.75 | 4.75 | 4.75 | 4.75 |
| turpentine oil (% w/w) | 4.75 | 4.75 | 4.75 | 4.75 | 4.75 | 4.75 | 4.75 | 4.75 | 4.75 |
| camphor oil (% w/w) | 4.75 | — | 4.75 | 4.75 | 4.75 | 2.375 | — | 4.75 | 2.375 |
| menthol (% w/w) | — | 4.75 | — | — | — | 2.375 | 4.75 | — | 2.375 |
| sassafras oil (% w/w) | 4.75 | 4.75 | 4.75 | 4.75 | 4.75 | 4.75 | 4.75 | 4.75 | — |
| beta carotene* (% w/w) | 4.75 | 4.75 | 4.75 | 4.75 | 4.75 | 4.75 | 4.75 | 4.75 | 4.75 |
| emu oil (% w/w) | 1 | 1 | 1 | 1 | 1 | 1 | 1 | — | 1 |
| oleoresin capsicum (% w/w) | 0.59 | 0.59 | 0.59 | 0.59 | 0.59 | 0.59 | 0.59 | 0.59 | 0.59 |
| chlorophyllin (% w/w) | — | — | 0.3 | — | 0.3 | 0.3 | 0.3 | — | — |
| mustard oil (% w/w) | — | — | — | 0.03 | 0.03 | 0.03 | 0.03 | — | — |
| medium wax (% w/w) | 4.79 | 4.79 | 4.79 | 4.79 | 4.79 | 4.79 | 4.79 | 4.79 | 4.79 |
| white petrolatum (% w/w) | 69.87 | 69.87 | 69.57 | 69.84 | 69.54 | 69.54 | 69.54 | 70.87 | 74.62 |

*30% w/win oil

II. Analgesic Compositions that can be Administered by Spraying.

| | Composition | | | | | | | | |
|---|---|---|---|---|---|---|---|---|---|
| Component | 1 | 2 | 3 | 4 | 5 | 6 | 7 | 8 | 9 |
| methyl salicylate (% w/w) | 4.75 | 4.75 | 4.75 | 4.75 | 4.75 | 4.75 | 4.75 | 4.75 | 4.75 |
| turpentine oil (% w/w) | 4.75 | 4.75 | 4.75 | 4.75 | 4.75 | 4.75 | 4.75 | 4.75 | 4.75 |
| camphor oil (% w/w) | 4.75 | — | 4.75 | 4.75 | 4.75 | 2.375 | — | 4.75 | 2.375 |
| menthol (% w/w) | — | 4.75 | — | — | — | 2.375 | 4.75 | — | 2.375 |
| sassafras oil (% w/w) | 4.75 | 4.75 | 4.75 | 4.75 | 4.75 | 4.75 | 4.75 | 4.75 | — |
| beta carotene* (% w/w) | 4.75 | 4.75 | 4.75 | 4.75 | 4.75 | 4.75 | 4.75 | 4.75 | 4.75 |
| emu oil (% w/w) | 1 | 1 | 1 | 1 | 1 | 1 | 1 | — | 1 |
| oleoresin capsicum (% w/w) | 0.59 | 0.59 | 0.59 | 0.59 | 0.59 | 0.59 | 0.59 | 0.59 | 0.59 |
| chlorophyllin (% w/w) | — | — | 0.3 | — | 0.3 | 0.3 | 0.3 | — | — |
| mustard oil (% w/w) | — | — | — | 0.03 | 0.03 | 0.03 | 0.03 | — | — |
| polysorbate 80 | 5 | 5 | 5 | 5 | 5 | 5 | 5 | 5 | 5 |
| glycerin | 20 | 20 | 20 | 20 | 20 | 20 | 20 | 20 | 20 |
| poloxamer 188 | 20 | 20 | 20 | 20 | 20 | 20 | 20 | 20 | 20 |
| SD alchohol 40-B | 15 | 15 | 15 | 15 | 15 | 15 | 15 | 15 | 15 |
| water | 14.66 | 14.66 | 14.36 | 14.63 | 14.33 | 14.33 | 14.33 | 15.66 | 19.41 |

*30% w/w in oil

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A topical analgesic composition comprising 2%-6% methyl salicylate, 2%-6% camphor oil, 2%-6% turpentine oil, 2%-6% sassafras oil, 0.1%-5% oleoresin capsicum, 0.05%-1% chlorophyllin, and 0.1%-5% emu oil, wherein all percentages are by weight, based on the total weight of the composition.

2. The topical analgesic composition according to claim 1, further comprising 0.01%-0.1% mustard oil, and 2%-6% beta carotene.

3. The topical composition of claim 1, further comprising a pharmaceutically acceptable carrier.

4. The topical analgesic composition according to claim 3, wherein the carrier comprises white petrolatum and/or medium wax.

5. The topical analgesic composition according to claim 4, wherein the carrier comprises 2%-6% medium wax, and 60%-80% white petrolatum.

6. The topical analgesic composition of claim 3, wherein the pharmaceutically acceptable carrier comprises polysorbate, glycerin, oloxamer 188, alcohol, and/or water.

7. The topical analgesic composition according to claim 1 comprising 4.75% methyl salicylate, 4.75% camphor oil, 4.75% turpentine oil, 4.75% sassafras oil, 0.59% oleoresin capsicum, 0.3% chlorophyllin, and 1% emu oil.

8. The topical analgesic composition according to claim 7 further comprising 0.03% mustard oil, and 4.75% beta carotene.

9. The topical analgesic composition according to claim 8, further comprising 4.79% medium wax, and 69.54% white petrolatum.

10. A topical analgesic composition according to claim 1, consisting of 4.75% methyl salicylate, 4.75% turpentine oil, 4.75% camphor oil, 4.75% sassafras oil, 4.75% beta carotene, 1% emu oil, 0.59% oleoresin capsicum, 0.3% chlorophyllin, 0.03% mustard oil, 4.79% medium wax, and 69.54% white petrolatum.

11. A topical analgesic composition according to claim 1, consisting of 4.75% methyl salicylate, 4.75% turpentine oil, 4.75% menthol, 4.75% sassafras oil, 4.75% beta carotene, 1% emu oil, 0.59% oleoresin capsicum, 0.3% chlorophyllin, 0.03% mustard oil, 4.79% medium wax, and 69.54% white petrolatum.

12. A topical analgesic composition according to claim 1, consisting of 4.75% methyl salicylate, 4.75% turpentine oil, 4.75% sassafras oil, 4.75% beta carotene, 2.375% camphor oil, 2.375 menthol, 1% emu oil, 0.59% oleoresin capsicum, 0.3% chlorophyllin, 0.03% mustard oil, 4.79% medium wax, and 69.54% white petrolatum.

13. A method for soothing body aches comprising administering an analgesic composition topically at a location of a body ache to a subject in need thereof, wherein the subject experiences the body ache or anticipates a development of body ache, wherein the analgesic composition is a topical composition according to claim 1.

14. A method according to claim 13, wherein the analgesic composition consists of methyl salicylate, turpentine oil, camphor oil, sassafras oil, beta carotene, emu oil, oleoresin capsicum, chlorophyllin, mustard oil, medium wax, and white petrolatum.

15. A method according to claim 13, wherein the analgesic composition consists of 4.75% methyl salicylate, 4.75% turpentine oil, 4.75% camphor oil, 4.75% sassafras oil, 4.75% beta carotene, 1% emu oil, 0.59% oleoresin capsicum, 0.3% chlorophyllin, 0.03% mustard oil, 4.79% medium wax, and 69.54% white petrolatum.

16. The method according to claim 13, wherein the subject has engaged in strenuous physical activity or is about to engage in strenuous activity.

17. The method according to claim 16, wherein the strenuous activity is weight lifting or participation in sports.

18. A therapeutic composition comprising 10%-45% active ingredients, and 55%-90% of pharmaceutical carriers, wherein the active ingredients comprise methyl salicylate, turpentine oil, camphor oil, menthol, sassafras oil, beta carotene, emu oil, oleoresin capsicum, chlorophyllin, and mustard oil.

19. The therapeutic composition according to claim 18, wherein the concentrations of the active ingredients are 2%-6% methyl salicylate, 2%-6% turpentine oil, 2%-6% camphor oil, 2%-6% menthol, 2%-6% sassafras oil, 2%-6% beta carotene, 0.05%-2% emu oil, 0.1%-2% oleoresin capsicum, 0.05%-1% chlorophyllin, and 0.01%-1% mustard oil.

20. The therapeutic composition according to claim 18, wherein the pharmaceutical carriers comprise 50%-85% white petrolatum and 2%-6% medium wax.

21. The therapeutic composition according to claim 18, wherein the pharmaceutical carriers are selected from the group consisting of polysorbate 80, glycerin, poloxamer 188, SD alcohol 40-B, water, and mixtures thereof.

22. An analgesic composition according to claim 18, consisting of 4.75% methyl salicylate, 4.75% turpentine oil, 4.75% camphor oil, 4.75% sassafras oil , 4.75% beta carotene, 1% emu oil, 0.59% oleoresin capsicum, 0.3% chlorophyllin, 0.03% mustard oil, 5% polysorbate 80, 20% glycerin, 20% poloxamer 188, 15% S SD alcohol 40-B, and 14.33% water.

23. An analgesic composition comprising methyl salicylate, turpentine oil, sassafras oil, beta carotene, emu oil, oleoresin capsicum, and at least one of camphor oil and menthol.

24. The analgesic composition according to claim 23, further comprising chlorophyllin and/or mustard oil.
